(19) United States
(12) Plant Patent Application Publication
(10) Pub. No.: US 2011/0307985 P1
(43) Pub. Date: Dec. 15, 2011

(54) Phlox plant named 'Barsixtythree'

(50) ;

(75) Inventor: Gosen B.H. Bartels, Aalsmeer (NL)

(73) Assignee: Bartels Breeding B.V., Aalsmeer (NL)

(21) Appl. No.: 12/802,686

(22) Filed: Jun. 11, 2010

(51) Int. Cl. A01H 5/00

(52) U.S. Cl. PLT/320

(57) ABSTRACT

A new Phlox paniculata plant particularly distinguished by a vigorous growth habit and purple inflorescences is disclosed.

### GENUS AND SPECIES

Phlox paniculata

### VARIETY DENOMINATION

‘Barsixtythree’

### BACKGROUND OF THE NEW PLANT

The present invention comprises a new and distinct variety botanically known as Phlox paniculata, and hereinafter referred to by the variety name ‘Barsixtythree’. The new variety originated from a crossing made in 2003 in Aalsmeer, The Netherlands. The female parent was the proprietary Phlox paniculata plant ‘00-48-02’ (unpatented), while the male parent was the proprietary Phlox paniculata plant ‘00-77-01’ (unpatented).

‘Barsixtythree’ was first propagated by vegetative cuttings in 2003 in Aalsmeer, The Netherlands and has been subsequently asexually propagated by vegetative cuttings in Aalsmeer, The Netherlands since 2003. The present invention has been found to retain its distinctive characteristics through successive asexual propagations via vegetative cuttings.

Plant Breeder's Rights for this variety were applied for in May 2010 for Europe. ‘Barsixtythree’ has not been made publicly available or sold more than one year prior to the filing date of this application.

### SUMMARY OF THE INVENTION

The following are the most outstanding and distinguishing characteristics of this new variety when grown under normal horticultural practices in Aalsmeer, The Netherlands.

1. A vigorous growth habit; and

2. Purple inflorescences.

### DESCRIPTION OF THE PHOTOGRAPHS

This Phlox paniculata plant is illustrated by the accompanying photographs which show the overall plant habit including blooms, buds, and foliage of the plant. The colors shown are as true as can be reasonably obtained by conventional photographic procedures.

FIG. 1 shows the overall plant habit.

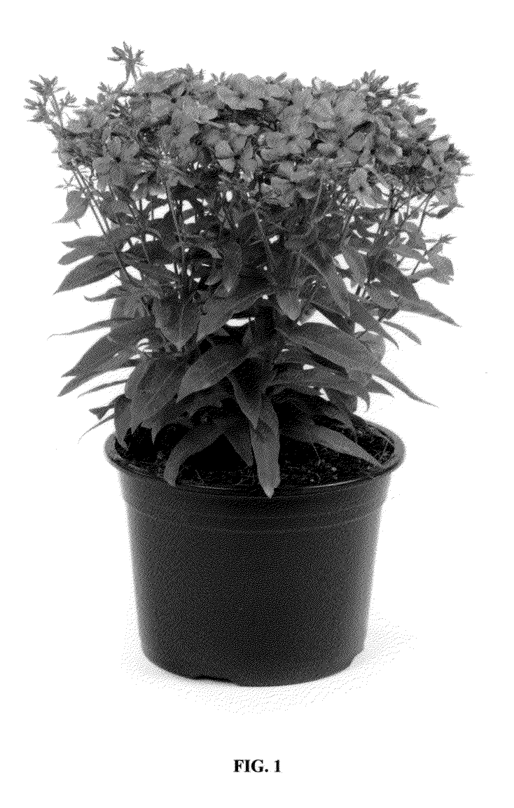

FIG. 2 shows a close-up of an inflorescence.

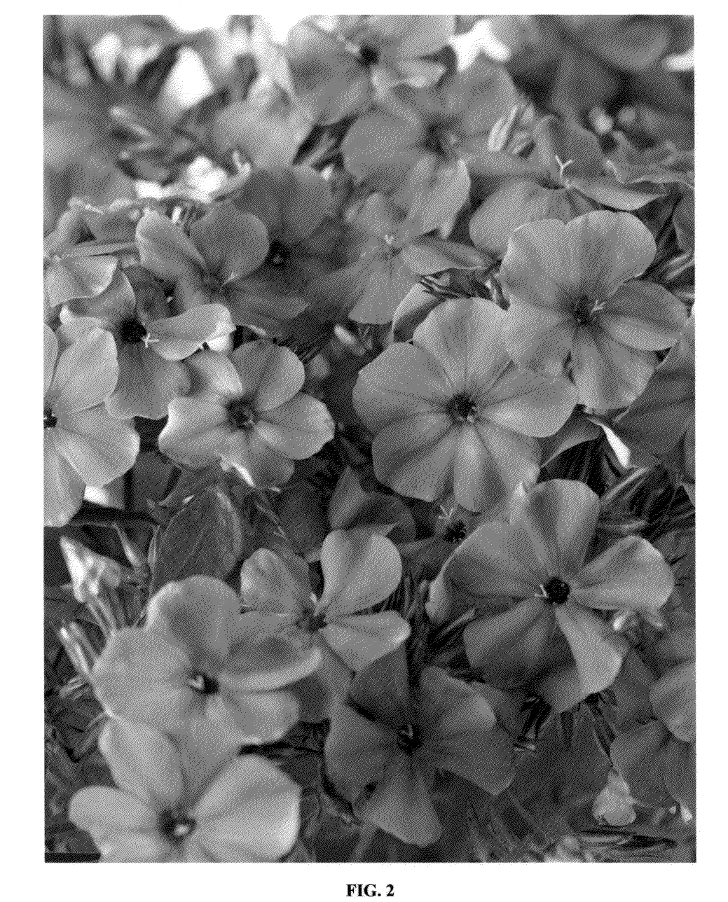

### DESCRIPTION OF THE NEW VARIETY

The following detailed description sets forth the distinctive characteristics of ‘Barsixtythree’. The data which defines these characteristics were collected from asexual reproductions carried out in Aalsmeer, The Netherlands in spring/early summer 2010. Color readings were taken under natural light outdoors. Color references are primarily to the R.H.S. Colour Chart of The Royal Horticultural Society of London (R.H.S.) (2001).

### DETAILED BOTANICAL DESCRIPTION

Classification:
   Family.—Polemoniaceae.
   Botanical name.—Phlox paniculata.
   Common name.—Phlox.
   Denomination.—‘Barsixtythree’.
Plant:
   Growth habit.—Vigorous.
   Form.—Narrow, bushy.
   Height (measured from the top of the soil).—30.0 cm to 40.0 cm.
   Diameter or area of spread.—25.0 cm.
   Propagation.—Via vegetative cuttings.
   Time to produce a finished flowering plant.—12 weeks.
   Lastingness of inflorescence on the plant.—3 weeks.
   Outdoor plant performance.—Good.
   Time to initiate and develop roots.—2 weeks.
   Time to produce rooted cutting or liner (young plant).—5 weeks.
   Root description.—Fleshy.
Branches:
   Number of branches per plant.—3 to 5.
   Length of lateral branches.—30.0 cm.
   Diameter of lateral branches.—15.0 cm.
Stems:
   Shape in cross-section.—Round.
   Color.—RHS 137B.
Inflorescence:
   Inflorescence type.—Self-cleaning.
   Arrangement.—Terminal and lateral.
   Petals.—Arrangement: Single whorl.
   Calyx.—Arrangement: Single whorl.

### COMPARISON WITH COMMERCIAL VARIETY

‘Barsixtythree’ differs from the commercial variety ‘Barfourteen’ (U.S. Plant Pat. No. 12,605) in that ‘Barsixtythree’ has a flat flowering canopy, smaller-sized flowers (1.5 cm), and an earlier flowering time, while ‘Barfourteen’ has a raceme-like flowering canopy, larger-sized flowers (2.0 cm to 3.0 cm), and the flowering time is two to three weeks later than ‘Barsixtythree’.

I claim:

1. A new and distinct variety of Phlox paniculata plant as described and shown herein.
